(19) United States
(12) Patent Application Publication
(10) Pub. No.: US 2025/0121117 A1
(43) Pub. Date: Apr. 17, 2025

(54) ADDITIVELY-MANUFACTURED COMPOSITE ROD FOR SPINAL INSTRUMENTATION

(71) Applicant: Paul E. Kraemer, Westfield, IN (US)

(72) Inventor: Paul E. Kraemer, Westfield, IN (US)

(21) Appl. No.: 18/917,719

(22) Filed: Oct. 16, 2024

Related U.S. Application Data
(60) Provisional application No. 63/590,652, filed on Oct. 16, 2023.

(51) Int. Cl. A61L 27/34; A61B 17/00; A61B 17/70; A61B 90/00; A61L 27/30

(52) U.S. Cl. CPC A61L 27/34; A61B 17/7002; A61L 27/306; A61B 2017/00526; A61B 2017/00964; A61B 2090/3966; A61L 2400/18; A61L 2420/04; A61L 2430/02

(57) ABSTRACT

A composite rod for spinal instrumentation may include a metal rod forming an inner core; and a composite polymer derived from a hybrid of PEEK and negatively (−) charged zeolite molecules forming an outer coating around at least a top portion of the inner core. The inner core and the outer coating each have a variable thickness, thereby forming a composite rod having a uniform overall thickness. The composite rod may be 3D-printed and may optimize fusion via rod stiffness and osteoconductive matrix spanning the levels fused, while maximizing pre-operative customization and surgeon intra-operative flexibility.

### CROSS-REFERENCE TO RELATED APPLICATION

The present application is a non-provisional of, and claims priority to, U.S. Patent Application No. 63/590,652 filed Oct. 16, 2023, the disclosure of which is incorporated by reference in its entirety.

### FIELD OF THE DISCLOSURE

The present disclosure generally relates to rods for spinal instrumentation, and more particularly, to additively-manufactured (e.g., 3D-printed) composite rods for spinal instrumentation that may optimize fusion via rod stiffness and osteoconductive matrix spanning of the levels fused, while maximizing pre-operative customization and surgeon intra-operative flexibility.

### BACKGROUND

Posterior instrumented thoracolumbar spinal fusion is a common and generally successful operation on the spine. Despite a long history, there continue to be rapid advancements in the hardware used to perform such operations.

Current challenges in such procedures include optimal fusion, optimal bone graft continuity, and particularly optimal stiffness. What is optimal at one area of the spine, for example the lumbosacral junction, may not necessarily be optimal at another area of the spine, such as the thoracolumbar (TL) junction or above. This necessitates making available a substantial number and variety of spinal fusion rods of variable stiffness profiles for various needs.

Current fusion rod materials may be customized, and can vary in flexibility, but are generally uniform across the material and are minimally osteoconductive. Alternative materials, such as polyether ether ketone (PEEK) are not osteoconductive at all and offer no intra-operative flexibility such as bending or cutting.

### SUMMARY

Embodiments of the present disclosure may provide a composite rod for spinal instrumentation including: a metal rod forming an inner core; and a composite polymer derived from a hybrid of PEEK and negatively (−) charged ceramic aluminum silicate molecules forming an outer coating around at least a first portion of the inner core, wherein the inner core and the outer coating each may have a variable thickness, thereby forming the composite rod of uniform overall thickness. The composite rod may be formed through 3D printing or other additive manufacturing techniques. The inner core may be formed of chrome cobalt or titanium alloy. The inner core may have a diameter up to about 6 millimeters. The inner core may have a diameter of about 5.5 millimeters. The outer coating may have a diameter up to about 6 millimeters. The composite rod may be capable of use in screw capture and/or a scaffolding function for osteocytes to travel along to promote fusion. The composite rod may be formed through plasma coating or machining. The inner core may have at least one portion including the outer coating. At least some portion of the composite rod may be metal only or the composite polymer only. Segmental stiffness, bend, and/or length of the composite rod may be customizable. The composite rod may provide improved radiopacity.

Other embodiments of the present disclosure may provide a composite rod for spinal instrumentation including: a metal rod forming an inner core; and a composite polymer derived from a hybrid of PEEK and negatively (−) charged ceramic zeolite molecules forming an outer coating around at least a first portion of the inner core, wherein the inner core and the outer coating each have a variable thickness, thereby forming the composite rod of uniform overall thickness. The negatively (−) charged ceramic zeolite molecules may be negatively (−) charged ceramic aluminum silicate molecules. No positively (+) charged heavy metal ion may be included as part of the outer coating. The composite rod may be formed through an additive manufacturing technique such as 3D printing, plasma coating, or machining. The inner core may have a diameter up to about 6 millimeters. The outer coating may have a diameter up to about 6 millimeters.

Further embodiments of the present disclosure may provide a composite rod for spinal instrumentation including: a metal rod forming an inner core; and a composite polymer derived from a hybrid of PEEK and negatively (−) charged ceramic zeolite molecules forming an outer coating around at least a first portion of the inner core, wherein the inner core and the outer coating each have a variable thickness, thereby forming the composite rod of uniform overall thickness, and wherein the composite rod may be formed through additive manufacturing, such as 3D printing, or another suitable manufacturing technique.

### BRIEF DESCRIPTION OF THE DRAWINGS

For a more complete understanding of this disclosure, reference is now made to the following description, taken in conjunction with the accompanying drawings, in which:

FIG. 1 is a perspective view of a composite rod according to an embodiment of the present disclosure.

FIG. 2A is a cross-section of the composite rod of FIG. 1 taken along line A-A.

FIG. 2B is a cross-section of the composite rod of FIG. 1 taken a long line B-B.

### DETAILED DESCRIPTION

Embodiments of the present disclosure may provide 3D-printed composite rods for spinal instrumentation that may optimize fusion via rod stiffness and osteoconductive matrix spanning the levels fused, while maximizing pre-operative customization and surgeon intra-operative flexibility. More specifically, rods may be additively manufactured (e.g., 3D-printed) and formed of a composite polymer derived from a hybrid of PEEK and negatively (−) charged zeolite molecules. One suitable such material is ZFUZE™ composite polymer (DiFUSION Technologies, Inc.; Austin, TX), which is a composite polymer derived from PEEK and negatively (−) charged ceramic aluminum silicate molecules. It should be appreciated, however, that other forms of negatively charged zeolites may be compounded with the PEEK polymer without departing from the present disclosure.

Regardless of what zeolite may be used as part of the hybrid composite polymer, it should be appreciated that, in some embodiments of the present disclosure, the composite polymer is devoid of any positively (+) charged heavy metal ion.

The hybrid composite polymer may be a hydrophilic load-bearing medical polymer that may provide for bony ingrowth into a spinal implantation and may afford the surgeon the ability to verify fusion radiographically.

Additively manufactured composite rods according to embodiments of the present disclosure may have a variable thickness metal core matched with a variable thickness hybrid composite polymer coating to provide a rod having a uniform overall thickness (e.g., in portions where the metal core is thinnest the coating will be thickest, and vice versa). These rods may utilize advances in pre-surgical planning and rod custom bending as well as material advances, including, but not limited to, the ability to 3D-print or otherwise additively manufacture the hybrid composite polymer.

The variable thickness metal core may be formed of one or more materials including, but not limited to, chrome cobalt or titanium alloy; however, other alloys may be used without departing from the present disclosure. The metal core may be constructed and bent with a diameter that varies along its length. It should be appreciated that a thicker diameter may lead to greater stiffness and less give in embodiments of the present disclosure.

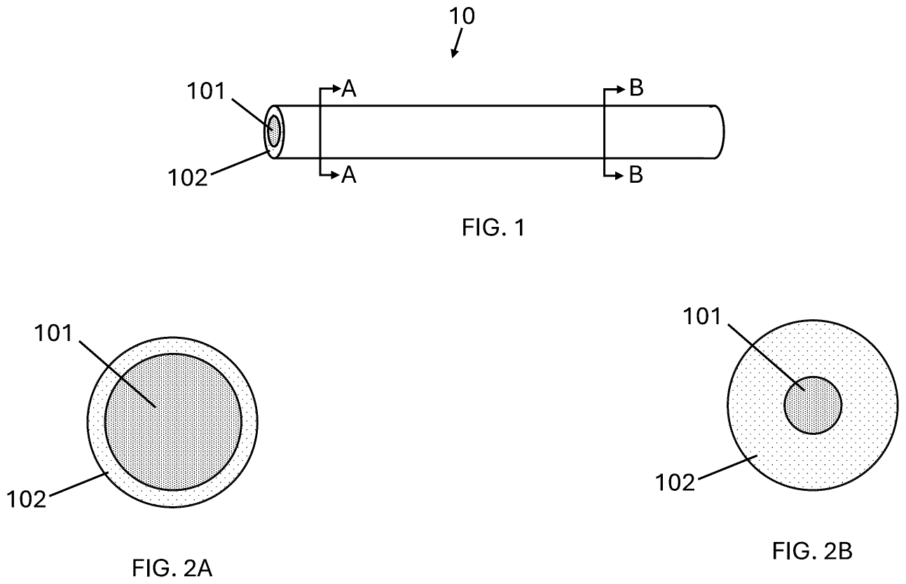

FIG. 1 depicts a composite rod 10 according to an embodiment of the present disclosure. As illustrated to good advantage in FIGS. 2A and 2B, composite rod 10 may be formed of an inner metal core 101 and an outer coating 102. While certain thicknesses are depicted in the Figures, it should be appreciated that these thicknesses are merely illustrative and that the thicknesses of the inner metal core and outer coating may be varied such that composite rod 10 has a uniform overall thickness in embodiments of the present disclosure.

Thus, as shown in FIGS. 2A and 2B, outer coating 102, which may be a hybrid composite polymer as described above, may be coated with varying thickness over inner metal core 101, which itself varies in thickness along its length, such that composite rod 10 has a uniform overall thickness. In other words, outer coating 102 may have a smaller diameter where metal core 101 has a larger diameter and vice versa. This may allow for appropriate rod capture in embodiments of the present disclosure.

As composite rod 10 according to the present disclosure may have less metal, it may exhibit lower stiffness and greater flexibility. By varying the respective thicknesses of metal core 101 and outer coating 102, these properties may be adjusted for a given procedure based on factors including, but not limited to, fusion length, bone strength, and degree of instability.

Because the hybrid composite polymer utilized in outer coating 102 is flexible, it cannot be plastically deformed intraoperatively. Accordingly, outer coating 102 may be constructed and formed on metal core 101 based on a preoperative plan.

On the other hand, because the metal core 101 can be plastically deformed, composite rod 10 exhibits a limited degree of intraoperative customization via bending. Interaoperative customization may also include cutting composite rod 10 to a desired length.

It should be appreciated that composite rod 10 may have an overall diameter of about 5.5 millimeters in embodiments of the present disclosure. However, there may be embodiments of the present disclosure where composite rod 10 may have an overall diameter of up to about 6 millimeters. Accordingly, the overall diameter of composite rod 10 may be up to about 6 millimeters, with metal core 101 forming the inner portion of composite rod 10 and outer coating 102 forming the outer portion of composite rod 10.

Outer coating 102 may have a diameter of up to about 6 mm in embodiments of the present disclosure. Outer coating 102 may have a thickness sufficient not to flake off in use. In an embodiment of the present disclosure, composite rod 10 may be formed entirely of a PEEK/zeolite composite polymer; however, embodiments of the present disclosure generally may include a metal core 101 and a hybrid composite polymer outer coating 102.

Composite rod 10 according to embodiments of the present disclosure may provide uniformity of diameter for functions including, but not limited to, screw capture, scaffolding function for osteocytes to travel along to promote fusion, and flexibility of rod for optimal stiffness. The underlying metal core 101 according to embodiments of the present disclosure may provide greater stiffness, radiopacity, and the ability to provide some ability to bend composite rod 10 intraoperatively.

While a 3D-printed hybrid composite polymer outer coating 102 has been described above, there may be some embodiments where outer coating 102 may be a hybrid of the hybrid composite polymer and metal. In such embodiments, the metal may be of variable stiffness. There may be alternative embodiments utilizing a more traditional PEEK coating. In some embodiments of the present disclosure, it should be appreciated that outer coating 102 may only cover a portion of metal core 101. In such embodiments, at least some portion of the composite rod 10 may be purely metal (e.g., the full overall diameter of composite rod 10 corresponds to that of metal core 101) or purely hybrid composite polymer (e.g., the full overall diameter of composite rod 10 corresponds to that of outer coating 102). However, it should be appreciated that there may be at least one portion of the length of composite rod 10 that contains both metal core 101 and outer coating 102 in embodiments of the present disclosure. Further, there may be some embodiments of the present disclosure where plasma coating or machining may be employed to form the composite rods instead of 3D-printing.

3D-printed composite rods for spinal instrumentation according to embodiments of the present disclosure may provide a number of benefits/advantages. For example, segmental stiffness may be customized for optimal mechanics at each portion of the spine. An osteoconductive scaffold may be provided due to the 3D-printed composite polymer coating. Bend and length may be customized in embodiments of the present disclosure. There also may be seamless integration of current screws and tools, as well as preoperative templating software/pre-bent rods. Improved radiopacity also may be provided in some embodiments of the present disclosure.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A composite rod for spinal instrumentation comprising:
a metal rod forming an inner core; and
a composite polymer derived from a hybrid of PEEK and negatively (−) charged ceramic aluminum silicate molecules forming an outer coating around at least a top portion of the inner core,
wherein the inner core and the outer coating each have a variable thickness and the composite rod has a uniform overall thickness.

2. The composite rod of claim 1, wherein the composite rod is formed through 3D printing.

3. The composite rod of claim 1, wherein the inner core is formed of chrome cobalt or titanium alloy.

4. The composite rod of claim 1, wherein the inner core has a diameter up to 6 millimeters.

5. The composite rod of claim 1, wherein the inner core has a diameter of 5.5 millimeters.

6. The composite rod of claim 1, wherein the outer coating has a diameter up to 6 millimeters.

7. The composite rod of claim 1, wherein the composite rod is capable of use in screw capture.

8. The composite rod of claim 1, wherein the composite rod is capable of use in a scaffolding function for osteocytes to travel along to promote fusion.

9. The composite rod of claim 1, wherein the composite rod is formed through plasma coating or machining.

10. The composite rod of claim 1, wherein the inner core has at least one portion including the outer coating.

11. The composite rod of claim 1, wherein at least some portion of the composite rod is metal only or the composite polymer only.

12. The composite rod of claim 1, wherein segmental stiffness, bend, and/or length of the composite rod are customizable.

13. The composite rod of claim 1, wherein the composite rod provides improved radiopacity.

14. A composite rod for spinal instrumentation comprising:
a metal rod forming an inner core; and
a composite polymer derived from a hybrid of PEEK and negatively (−) charged ceramic zeolite molecules forming an outer coating around at least a top portion of the inner core,
wherein the inner core and the outer coating each have a variable thickness and the composite rod has a uniform thickness.

15. The composite rod of claim 14, wherein the negatively (−) charged ceramic zeolite molecules are negatively (−) charged ceramic aluminum silicate molecules.

16. The composite rod of claim 14, wherein no positively (+) charged heavy metal ion is included as part of the outer coating.

17. The composite rod of claim 14, wherein the composite rod is formed through 3D printing, plasma coating, or machining.

18. The composite rod of claim 14, wherein the inner core has a diameter up to 6 millimeters.

19. The composite rod of claim 14, wherein the outer coating has a diameter up to 6 millimeters.

20. A composite rod for spinal instrumentation comprising:
a metal rod forming an inner core; and
a composite polymer derived from a hybrid of PEEK and negatively (−) charged ceramic zeolite molecules forming an outer coating around at least a top portion of the inner core,
wherein the inner core and the outer coating each have a variable thickness and the composite rod has a uniform thickness, and
wherein the composite rod is formed through 3D printing.
